(19) United States
(12) Patent Application Publication
(10) Pub. No.: US 2017/0156728 A1
(43) Pub. Date: Jun. 8, 2017

(54) SURGICAL FASTENERS WITH INDICATION OF FASTENER AMOUNT

(71) Applicant: Filippo Bastia, Sozzigalli (MO) (IT)

(72) Inventor: Filippo Bastia, Sozzigalli (MO) (IT)

(21) Appl. No.: 14/956,487

(22) Filed: Dec. 2, 2015

(51) Int. Cl. A61B 17/072; A61B 17/00; A61B 17/08; A61B 17/10; A61B 90/92

(52) U.S. Cl. CPC A61B 17/072; A61B 17/105; A61B 90/92; A61B 17/07207; A61B 17/083; A61B 17/00234; A61B 2017/07235; A61B 2090/0803

(57) ABSTRACT

A plurality of surgical fasteners includes a first-to-be-applied fastener, at least one next-to-be-applied fastener and a last-to-be-applied fastener. At least one of the fasteners is visibly and recognizably different than the other surgical fasteners.

### FIELD OF THE INVENTION

The present invention relates generally to surgical fasteners, such as for use with tacking devices or other fastener applying devices, and particularly to surgical fasteners that provide an indication when the fastener applying device has reached the last fastener or fasteners or any other indication of an amount of fasteners.

### BACKGROUND OF THE INVENTION

Some surgical procedures involve delivering a series of clips such as tacks, staples, or sutures to a surgical site within a patient. For example, hernia repair typically involves a laparoscopic procedure which uses a surgical tacking device to deliver a series of clips or tacks to fix a hernia mesh in place.

However, if the tacking device runs out of fasteners during the procedure, the mesh can go astray in an unpredictable manner. The mesh may fold or flop into positions that are unmanageable and may result in bowel adhesions and post-operative pain. Accordingly, it is desirable to know ahead of time when the tacking device is reaching its last fastener.

U.S. Pat. No. 5,356,064 describes a surgical stapler that includes a staple follower made of a bright colored plastic material such as nylon. For example, the head of the staple follower is fabricated of a bright yellow material such that a visible indication is provided by the head after the last staple has been spent. The colored indicator may be resilient and snap into place when the last staple has been spent.

U.S. Pat. No. 5,582,616 describes a surgical helical fastener applicator that has a lock clip indicator that applies a spring force on the fasteners. The lock clip/indicator serves as a jam stop to prevent further actuation by a rotator upon discharge of all the fasteners from the device. The lock/indicator can be made of a color (or shape) to serve as an empty indicator notifying the user that no more fasteners are available.

PCT Patent Application WO 2014/177939 describes a tacking device with a quantity indicator on a distal end that can be seen through a scope and that shows a number of fasteners remaining in the device. A surgeon can watch the delivery of fasteners while simultaneously seeing the number of fasteners remaining. The indicator can be a marker element disposed within the carrier portion and an electronic display (e.g., an LED), coupled to an endoscopic camera. The indicator could also be a color-coded portion of the tacking device, in which a progressive color change indicates the number of fasteners remaining (e.g., green is full; blue is partially full; red is almost empty (last 3 fasteners); and black is empty). The indicator is located at the very distal tip of the tacking device.

However, all of the abovementioned devices are not easily seen by the surgeon who is paying attention to the fasteners and tissues and not to the device. The prior art solutions require additional parts/cost and may degrade the device reliability.

### SUMMARY OF THE INVENTION

The present invention seeks to provide improved surgical fasteners that provide an indication when the fastener applying device has reached the last fastener or fasteners or any other indication of an amount of fasteners, as described in more detail further below. Unlike the prior art, in the present invention the indication is provided on the fastener and not on the fastener applying device. Thus, in the present invention as opposed to the prior art, the surgeon who is paying attention to the fasteners and tissues can easily discern when the fastener applying device has reached the last fastener or fasteners. The indication can be used to indicate the delivery progress and/or the end of the fasteners.

There is thus provided in accordance with an embodiment of the present invention a surgical fastener assembly including a plurality of surgical fasteners including a first-to-be-applied fastener, at least one next-to-be-applied fastener and a last-to-be-applied fastener, wherein at least one of the fasteners is visibly and recognizably different than others of the surgical fasteners. The at least one of the fasteners may be placed among the other surgical fasteners in a manner that indicates progressive use of the surgical fasteners.

The at least one of the fasteners may include the last-to-be-applied fastener. The at least one of the fasteners may include a plurality of fasteners placed at intervals among the surgical fasteners.

For example, the at least one fastener may have a different color than others of the surgical fasteners. Additionally or alternatively, the at least one fastener may have a different geometrical feature than others of the surgical fasteners or any other kind of mark. The different color may include a different level of radio-opacity.

In accordance with an embodiment of the present invention there are more than one next-to-be-applied fasteners, and some of the next-to-be-applied fasteners are visibly and recognizably different than others of the surgical fasteners.

In accordance with an embodiment of the present invention at least some of the next-to-be-applied fasteners are not visibly different than the last-to-be-applied fastener.

In accordance with another embodiment of the present invention at least some of the next-to-be-applied fasteners are visibly different than the last-to-be-applied fastener.

There is also provided in accordance with an embodiment of the present invention a method including providing a plurality of surgical fasteners including a first-to-be-applied fastener, at least one next-to-be-applied fastener and a last-to-be-applied fastener, wherein at least one of the fasteners is visibly and recognizably different than others of the surgical fasteners.

### BRIEF DESCRIPTION OF THE DRAWINGS

The present invention will be understood and appreciated more fully from the following detailed description, taken in conjunction with the drawings in which:

FIG. 1 is a simplified pictorial illustration of an array of surgical fasteners installed in a fastener applying device, constructed and operative in accordance with an embodiment of the present invention;

FIG. 2 is a simplified, more enlarged illustration of the surgical fasteners; and

FIG. 3 is a simplified pictorial illustration of one of the surgical fasteners with different coloring.

### DETAILED DESCRIPTION OF EMBODIMENTS

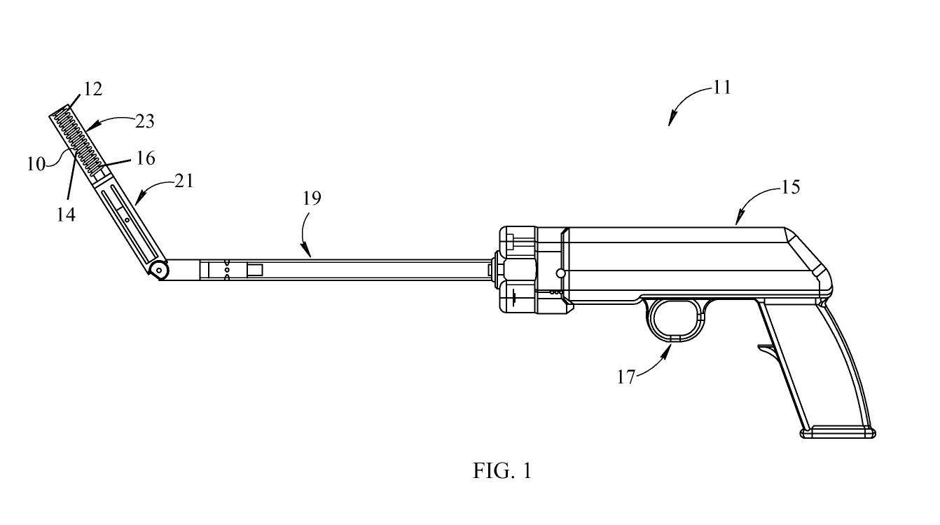

Reference is now made to FIG. 1, which illustrate an array of surgical fasteners 10 installed in a fastener applying device 11, constructed and operative in accordance with a non-limiting embodiment of the present invention.

The fastener applying device 11 shown in the drawings is the tacker of U.S. Pat. No. 8,006,365, but the fastener applying device is not limited to this tacker and may include, without limitation, a surgical tacker, suturing device, stapler or other devices.

The fastener applying device 11 may include a handle 15 with a trigger assembly 17. A drive shaft 19 is coupled to trigger assembly 17. An articulated applicator arm 21 is pivotally connected to drive shaft 19.

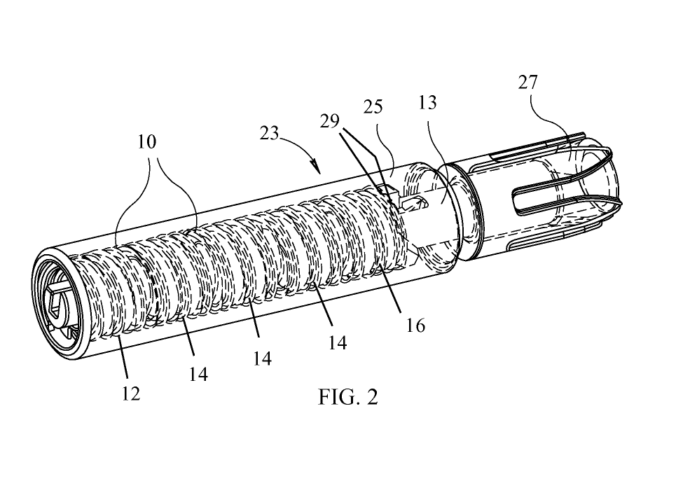

Reference is now made to FIG. 2, which illustrates a magazine 23, which is mounted in articulated applicator arm 21 and holds the array of surgical fasteners 10. Magazine 23 includes a housing 25 in which an applicator shaft 13 is journaled. A proximal end 27 of applicator shaft 13 is complimentarily shaped for mating with the drive shaft. One or more surgical fasteners 10 are disposed one after the other along applicator shaft 13 (five fasteners, each having three coils, are illustrated in the exemplary, non-limiting embodiment). Rotation of applicator shaft 13 advances surgical fasteners 10 off shaft 13 and screws or fastens them into tissue (not shown). The surgical fasteners 10 may include, without limitation, tacks, rotary tacks, helical tacks, staples, sutures and other fasteners. The invention is not limited to articulating devices and may be used with non-articulating devices, too. The fasteners do not have to be mounted in a magazine and may be mounted in a tube or other structure.

The plurality of surgical fasteners 10 includes a first-to-be-applied fastener 12, one or more next-to-be-applied fasteners 14 and a last-to-be-applied fastener 16. The terms “first-to-be-applied”, “next-to-be-applied” and “last-to-be-applied” refer to the order in which the fasteners are deployed from the fastener applying device. For example, the first-to-be-applied fastener 12 may be the most distal fastener, the one or more next-to-be-applied fasteners 14 may be proximal to the most distal fastener, and the last-to-be-applied fastener 16 may be the most proximal fastener.

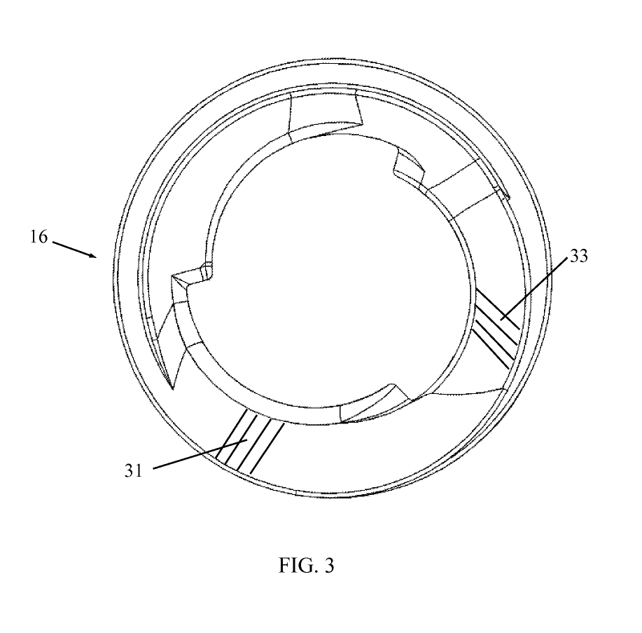

At least one of the fasteners, such as the last-to-be-applied fastener 16, may be visibly and recognizably different than at least some of the other surgical fasteners 12 and 14. For example, this fastener, such as the last-to-be-applied fastener 16, may have a different color than other fasteners. The different color may include a different pigment (e.g., red, yellow, blue, green, etc.), a different level of transparency (the last-to-be-applied fastener could be opaque while other fasteners could be transparent), or a different level of radio-opacity. This fastener, such as the last-to-be-applied fastener 16, may be completely colored or partially colored, such as stripes, dots and the like (as seen in FIG. 3 and indicated by reference numerals 31 and 33).

Additionally or alternatively, this fastener, such as the last-to-be-applied fastener, may have a different geometrical feature than other fasteners (such as being bent differently or shaped differently, as seen in FIG. 2 and designated by reference numeral 29). Additionally or alternatively, this fastener, such as the last-to-be-applied fastener, may have a different mark than other fasteners (such as a visible set of lines, which are not necessarily differently colored than other fasteners).

With the present invention, the surgeon can easily discern the last-to-be-applied fastener 16. In accordance with an embodiment of the present invention, the surgeon can be provided of an indication not just of the very last fastener, but also of the last few fasteners (such as the last 3 or 5 fasteners). Accordingly, as in the illustrated embodiment, there are more than one next-to-be-applied fasteners 14, and some of the next-to-be-applied fasteners 14 may be visibly and recognizably different than others of the surgical fasteners. Additionally or alternatively, at least some of the next-to-be-applied fasteners 14 may be visibly or not visibly different than the last-to-be-applied fastener 16.

Some of the fasteners can have one color (or shape or mark) and others can have another color (or shape or mark). The invention can be used to indicate progress of the fasteners in the device. For example, a differently colored (or shaped or marked) fastener can be placed every 10 fasteners (or other amount) to indicate the progress of the fasteners. Alternatively, one differently colored (or shaped or marked) fastener can be placed as the tenth fastener, 2 differently colored (or shaped or marked) fasteners can be placed as the 19^{th} and 20^{th} fasteners (or 20^{th} and 21^{st} fasteners, or 21^{st} and 22^{nd} fasteners), and 3 differently colored (or shaped or marked) fasteners can be placed as the 28^{th}, 29^{th} and 30^{th} fasteners (or 30^{th}, 31^{st} and 32^{nd} fasteners) and so forth. The indication serves as an index to keep track of the progress of the fasteners.

What is claimed is:

1. A surgical fastener assembly comprising:
a plurality of surgical fasteners comprising a first-to-be-applied fastener, at least one next-to-be-applied fastener and a last-to-be-applied fastener, wherein at least one of said fasteners is visibly and recognizably different than others of said surgical fasteners.

2. The assembly according to claim 1, wherein said at least one of said fasteners is placed among the other surgical fasteners in a manner that indicates progressive use of said surgical fasteners.

3. The assembly according to claim 1, wherein said at least one of said fasteners comprises said last-to-be-applied fastener.

4. The assembly according to claim 1, wherein said at least one of said fasteners comprises a plurality of fasteners placed at intervals among said surgical fasteners.

5. The assembly according to claim 1, wherein said at least one of said fasteners has a different color than others of said surgical fasteners.

6. The assembly according to claim 1, wherein said at least one of said fasteners has a different geometrical feature than others of said surgical fasteners.

7. The assembly according to claim 1, wherein said at least one next-to-be-applied fastener comprises a plurality of next-to-be-applied fasteners that are visibly and recognizably different than others of said surgical fasteners.

8. The assembly according to claim 7, wherein at least some of said next-to-be-applied fasteners are not visibly different than said last-to-be-applied fastener.

9. The assembly according to claim 7, wherein at least some of said next-to-be-applied fasteners are visibly different than said last-to-be-applied fastener.

10. The assembly according to claim 5, wherein said different color comprises a different level of radio-opacity.

11. A method comprising:
providing a plurality of surgical fasteners comprising a first-to-be-applied fastener, at least one next-to-be-applied fastener and a last-to-be-applied fastener, wherein at least one of said fasteners is visibly and recognizably different than others of said surgical fasteners and is placed among the other surgical fasteners in a manner that indicates progressive use of said surgical fasteners.
